(19) United States
(12) Patent Application Publication
(10) Pub. No.: US 2025/0049973 A1
(43) Pub. Date: Feb. 13, 2025

(54) IN-LINE RADIATION STERILIZATION FOR WOUND DRESSINGS

(71) Applicant: PAUL HARTMANN AG, Heidenheim (DE)

(72) Inventor: Christian Schmidt, Lauchheim (DE)

(21) Appl. No.: 18/722,249

(22) PCT Filed: Dec. 22, 2022
(86) PCT No.: PCT/EP2022/087409

§ 371 Date: Jun. 20, 2024

(30) Foreign Application Priority Data
Dec. 30, 2021 (EP) 21218354.5

(51) Int. Cl. A61L 2/08; A61F 15/00; A61L 2/24; A61L 2/26

(52) U.S. Cl. CPC A61L 2/087; A61F 15/001; A61L 2/24; A61L 2/26; A61L 2202/11; A61L 2202/121; A61L 2202/14; A61L 2202/23

(57) ABSTRACT

The present invention relates to an in-line method of radio sterilization of wound dressings using electrons. A packaged wound dressing is provided by means of a production apparatus and conveyed to a sterilization module by means of a conveying device. The packaged wound dressing is sterilized by means of a sterilization module coupled in-line to the production apparatus. The sterilization module comprises an electron beam source, which generates electrons having an energy of between 150 keV and 350 keV. The energy dose to which the packaged wound dressings are exposed is at least 15 kGy and no more than 30 kGy. The conveying device moves the packaged wound dressings at a speed of at least 10 m/min and no more than 200 m/min.

The present invention relates to an in-line method of radiosterilization of wound dressings using electrons.

As a rule, wound dressings are currently sterilized using ethyleneoxide. To this end, the individually packaged wound dressings are initially bound in boards and palletized. After preconditioning and sterilization, the sterilized goods must undergo sufficient ventilation to eliminate ethyleneoxide residues. Due to its complexity, this method is elaborate and expensive. Moreover, wound dressing sterilization is usually performed at sterilization centers, and so costs arise for the transport to and from these sterilization centers. In particular, the process, from shipping through preconditioning, sterilization, degassing, and quarantine, up to the return of the goods, is quite time-consuming. Cycle times of 10 days are usual. Alternatively, a sterilization by means of radiosterilization using gamma radiation is possible. For this purpose, a multiplicity of wound dressings packaged in cardboard and located on pallets are sterilized. Such a radio sterilization of wound dressings in palletized cardboard boxes using gamma radiation is possible at room temperature; however, the sterilization devices require a necessary shielding for the gamma radiation. The method requires radio active cobalt 60 as a source of radiation. The limited availability thereof and the complicated handling make this method very expensive.

In the prior art, electron beam sterilization is used for the packaging of surgical and pharmaceutical products. WO2009139073 describes a sterilization method by means of an electron beam, in which the radiation has only a comparatively low penetration depth. In this context, the intention is for the radiation to reach the inner side of the packaging material while the content of the packaging is protected from the damaging effect of the radiation at the same time.

WO02066081 relates to an electron beam irradiation device for sterilizing a sheet packaging material. The material is irradiated from two sides by an electron beam with an energy of no more than 200 keV.

The inventions with regards to radiosterilization present in the prior art relate to the sterilization of packaging in particular.

Moreover, high-energy electrons in an energy range from 7 MeV to 10 MeV are used in the prior art for the sterilization of secondarily (e.g., in a collapsible box) or tertiarily (in a shipping box) packaged medical products. This high energy is needed to be able to radiate through the entire packaging. Equipment operating with electrons in ranges with such high energies place very high demands on the shielding and are inconsistent, or very hard to make consistent, with the usual conditions in a wound dressing production apparatus.

There is a need for a reliable wound dressing sterilization method which can be performed at the production site.

The inventors of the present invention were confronted with the problem of improving the provision of sterile wound dressings. In particular, the inventors of the present invention were confronted with the problem of using ionizing rays to sterilize wound dressings already located in a sealed packaging. Specific requirements had to be considered here on account of the materials and packagings (sterile barrier system) used for wound dressings. Moreover, the problem addressed by the present invention is that of providing a sterilization method particularly suitable for wound dressings packaged in a primary packaging.

Theinventionsolvesthisproblembymeansofanin-linesterilizationmethodforwound dressings as claimed in claim 1. The method ensures an efficient in-line sterilization of the packaged wound dressing, which meets ISO 11137 specifications. The method is particularly suitable for wound dressings sealed in a primary packaging. In the present case, the
expression “in-line sterilization” is understood to mean a sterilization process which runs continuallydirectlyinthewounddressingproductionapparatusorwhichisperformed continually in a sterilization module functionally connected to this production apparatus. The sterilization is coupled to the wound dressing production process. Thus, “in-line” denotes coupling in both space and time. The sterilization module can be understood to be a component of a manufacturing line. The sterilization module throughput then corresponds to the manufacturing line throughput. The manufacturing line comprises all machines or modules contributing to the manufacture of the packaged sterile wound dressing. These manufacturing line modules comprise, inter alia, one or more production modules (i.e., machines required to manufacture a complete wound dressing from its source materials or an intermediate product),
a packaging machine (i.e., a machine which seals, i.e., packages, the wound dressings), a sterilization module (i.e., a device in which the packaged wound dressings are sterilized), and a conveying device. In the present case, the term “production apparatus” is understood to mean a device comprising the production module and the packaging machine.

The method comprises the following steps:
providing the packaged wound dressing by means of a production apparatus,
conveying the packaged wound dressings by means of a conveying device to a sterilization module,
sterilizing the packaged wound dressing by means of at least one electron beam source present in the sterilization module, wherein the sterilization module is coupled in-line to the production apparatus,
   a) and wherein the energy dose to which the packaged wound dressings are exposed is at least 15 kGy and no more than 30 kGy,
   b) and wherein the energy of the electron radiation is between 150 keV and 350 keV,
   c) and wherein the conveying device moves the packaged wound dressings at a speed of at least 10 m/min and no more than 200 m/min.

The production apparatus is a preferably automated machine arrangement, in which wound dressings can be produced using suitable source materials. For instance, the production apparatus comprises production modules which are configured for cutting the source materials usually present in material webs to size and for performing lamination processes. Likewise, the production apparatus comprises a preferably automated packaging machine which, in the production process, is connected downstream of the modules that produce the wound dressings.

A packaging machine particularly suitable for sterile packagings in particular is used. In the production process, wound dressings which are packaged in germproof fashion by the packaging machine are usually unsterile at first, since the entire production process runs in unsterile surroundings and the source materials are not provided in sterilized fashion either.

For example, in the case of an island dressing, the wound dressings manufactured by a production module comprise a wound contact layer, anabsorbentply (for example an absorbent pad), and a covering layer (“backing”). In this case, the wound contact layer comprises a smaller contact face than the covering layer, with the wound contact layer being surrounded by the covering layer along its circumference. In this case, the covering layer may have an adhesive coating wound side, with the result that the wound dressing is able to adhere or stick to the skin of a patient. A further embodiment of the wound dressing can be a wound contact layer with no further plies. Wound dressings, especially wound dressings with adhesive covering layers or adhesive edges, may contain further materials and layers for covering the
adhesive regions or for the back-side stabilization of the product, for example in the form of separation films (“liners”) or support films. For example, materials present in wound dressings comprisepolyethylene(PE), polypropylene(PP), polyethyleneterephthalate (PET), polyurethane (PU), viscose, cotton, silicone, polyacrylates, hydrocolloids, or ointments. What needs to be considered with regards to an electron beam-based sterilization is that the material properties (for instance adhesion, color, smell, tensile strength) may change under the action of the irradiation.

In the manufacturing process, the wound dressing production is followed by the wound dressing packaging. In the packaging machine, one or more wound dressings are packaged in a flexible pocket or in a pouch. By preference, only a single wound dressing is packaged in a respective flexible pocket or in a respective pouch. For example, the packaging machine canrelatetosystemsasdescribedinthepreviouslyunpublishedEuropeanPatentApplication EP20201966.7. Peel pouches consisting of an upper sterile barrier and a lower sterile barrier are one embodiment for the packaging. The sterile barriers are connected via an adhering
outer region so as to form an internal cavity for accommodating wound dressings. The upper and the lower sterile barrier consists of paper, film or a composite, wherein the upper sterile barrier material may differ from the lower sterile barrier material.

Thermoformed packagings can be a further example of sterile packagings.

In a preferred embodiment, the packagings are in a connected pouch chain when leaving the packaging machine. Thus, the packaging are connected to one another at their ends and therefore need to be cut to size in a subsequent process step in order to obtain individual packagings. The connected packagings and the wound dressings enclosed therein may have a web width b_{w} of between 0.02 m and 0.40 m. Web widths b_{w} between 0.05 m and 0.35 m are preferred; web widths b_{w} of between 0.1 m and 0.23 m are particularly preferred. The maximum height of the packaged wound dressings is preferably between 0.5 mm and 10.0 mm. Heights between 1.0 mm and 5.0 mm are more preferable. Heights between 1.5 and 2.5 mm are particularly preferred. The grammage of the connected, packaged wound dressings is at least 0.15 kg/m² and no more than 0.65 kg/m². By preference, the mean grammage of the connected, packaged wound dressings is at least 0.2 kg/m² and no more than 0.5 kg/m², more preferably between 0.2 kg/m² and 0.45 kg/m², and particularly preferably between 0.2 kg/m² and 0.35 kg/m². The grammage can be determined from the known grammage of the individual components of the wound dressings and their packaging. The conveying device is used to convey the connected pouch chain to the sterilization module via driven and non-driven rollers and/or conveyor belts, with or without vacuum assistance.

In another preferred embodiment, the connected pouch chain is cut to size into individual, separate packagings, which each contain one wound dressing, prior to the sterilization process. The individually packaged wound dressings are then conveyed to the sterilization module. The packaged wound dressings can also be held on a vacuum belt of the conveying device by means of negative pressure. In this case, the selection of material for the vacuum conveyor belt in the region of the sterilization module is attuned to a continual irradiation with the selected number of electrons having the selected energy. In a preferred embodiment, this is a chain link belt made of metal. This embodiment is particularly advantageous as it has very high radiation stability and is able to contribute to the sterilization by way of secondary electrons. Moreover, a metal chain link belt has good properties in relation to thermal conductivity. The pouches with the wound dressings situated there in are held securely in position by means of the vacuum conveyor belt as they are transported through the sterilization module. Thereafter, the pouches can preferably be transferred to a further, subsequently arranged module. In a possible embodiment, this is a further packaging machine, which stacks the individual pouches and packages these in a collapsible box (secondary packaging).

In another embodiment, the separation of the pouches into individual units is followed by the packaged wound dressing being transported by means of a conveying device which has ribs or elevations extending across the direction of transport. One packaged wound dressing is moved between two elevations. During transport, the elevations keep the wound dressings in the portions of the conveying device provided therefor.

In an alternative preferred embodiment, the individually packaged wound dressings can be held on the conveying device by means of pockets which are sealed or partially opened.

The conveying device moves the packaged wound dressings at a speed of at least v_{w}=10 m/min and no more than v_{w}=200 m/min. The conveying device more preferably moves the wound dressings at a speed of between v_{w}=20 m/min and v_{w}=200 m/min. The conveying device particularly preferably moves the wound dressings at a speed of between v_{w}=50 m/min and v_{w}=200 m/min. The conveying device most preferably moves the wound dressings at a speed of between v_{w}=100 m/min and v_{w}=200 m/min.

If the packaged wound dressings are transported in a connected pouch chain by means of the conveying device, then the area velocity is V_{F}=v_{w}·b_{w}, with the speed v_{w} and the width of the packaging b_{w}. The width of the packaging b_{w} is defined perpendicular to the conveying direction. The area velocity can be specified in m²/. The mass moved per minute is v=min m P_{G}·V_{F}, with the weight per square meter p_{G} of the packaged wound dressing in a connected pouch chain being given by kg/m², and

$$[ {v}_{m} ] = kg / min .$$

The area velocity is typically between 0,2ₘᵢₙ/m² and 80 ₘᵢₙ/m², while the mass moved per second is usually restricted to at least 0,03 kg/min and no more than 52 kg/min.

In an alternative embodiment, separated pouches are transported instead of the connected pouch chain, with a spacing of at least 1 mm usually present between the pouches. The conveying device speed needs to be increased in order to obtain a production speed comparable with the above-described embodiment (connected pouch chain). However, the electron beam source power must be increased when the conveying device speed is increased since electrons incident between the pouches do not contribute to the sterilization.

The sterilization module comprises at least one electron beam source and preferably at least one shielding device. Moreover, the sterilization module preferably comprises at least one guide for the conveying device.

By preference, the electron beam source is an electron accelerator. In the electron accelerator, electrons are emitted from an electron source, preferably by therm ion icemission from a cathode with preferably a low work function, and these electrons are accelerated by voltages. The electron accelerator and the electron beam source may contain further units suitable for conditioning or steering the electron beam.

In a preferred embodiment, the sterilization module is self-shielding, with the result that components present in the sterilization module, for instance the rollers of the conveying device and/or housing walls and/or further components, ensure sufficient radiation protection without further shielding, to be attached outside of the sterilization module, being necessary. Such a self-shielding sterilization module can be realized particularly advantageously, especially when using electron sterilizers that operate at lower electron energies (for example at 250 keV).

The sterilization module is preferably aligned in a continuation of a straight line in the transport direction of the conveying device present in the manufacturing line between production module and packaging machine. In another embodiment, the sterilization module is aligned orthogonal to this transport direction. To this end, the packaged wound dressings are preferably redirected in two steps, through 450 in each case, and guided through the sterilization module. Optionally, the wound dressings are redirected again downstream of the sterilization module, in such a way that they are conveyed in the original transport direction again. Two rotary rods at a 22.5° angle to the web direction are preferably used for moving a connected pouch chain.

PursuanttoDINENISO11137-1:2020-04, the energy dose of15 kGy received by the packaged wound dressings is considered safe for medical products. 30 kGy can be considered an upper limit for the energy dose. The upper limit arises for reasons of economy and, in many cases, material compatibility. In the case of wound dressings, adhesives in particular are found to be sensitive to high energy doses, under which they might lose their adhesive properties. Other examples of incompatibilities vis-à-vis energy doses greater than 30 kGy may include discoloration of plastics or an onset of packaging material brittleness.

The inventors of the present invention have discovered that an energy E of between 150 keV and 350 keV is particularly suitable for the irradiation of wound dressings. A lower bound of this energy range advantageously arises on account of the absorption of the radiation in the packaged wound dressing. Lower energy electrons do not appear to penetrate deep enough into the packaged wound dressing in order to develop a sterilization effect. Up to the lower energy bound of 150 keV proposed according to the invention, the undesirably strong absorption of the radiation in upper layers, which leads to an on uniform energy dose distribution in the wound dressing, can be compensated for by virtue of the arrangement of the sterilization module being chosen so that the products are irradiated from both sides, for example by two or more successive electron beam sources on opposite sides of the material web.

Energies above 350 keV are suitable for in-line sterilization of wound dressings as a matter of principle, but are not economical. As the energy increases, the mean free path length of the electrons becomes so large that a significant proportion of the radiation penetrates through the wound dressing and packaging without being absorbed. This proportion thus no longer contributes to the sterilization. Electrons having an energy of the order of several MeV, as already mentioned in the prior art, lead to significant outlay in the generation of the radiation. Furthermore, electron beam sources which generate very high energy electrons, especially those which generate an energy substantially above 350 keV, require complex shielding measures.

Energies between 280 keV and 350 keV are used in a further possible embodiment of the invention. This allows the use of those electrons which penetrate through the wound dressing without interacting with the wound dressing material. This is achieved by virtue of the material web being guided past the electron beam source multiple times, for example in loops.

The minimum power required for the sterilization module can be estimated by way of Pₛ=vₘ·D. D is the energy dose, which can be specified in kJ/kg. The power Pₛ is usually at least 100 Wand no more than 100 kW. In addition to the power losses when generating the electron beam, two effects, in particular, need to be considered when determining the actually required power: Firstly, a nonuniform distribution of the energy dose within the wound dressing and the wound dressing packaging should be assumed. A high dose uniformity ratio (DUR) pursuant to ISO 11137-3:2017 is an indicator of a nonuniform distribution. The DUR is defined as

$$DUR = \frac{{D}_{max}}{{D}_{min}} ,$$

where Dₘₐₓ is the maximum energy dose of the absorbed energy within the radiation container, and Dₘᵢₙ is the minimum energy dose of the absorbed energy within the radiation container. The radiation container is the packaged wound dressing.

Since the lowest energy dose measured on the sterilized wound dressing must be sufficient for sterilization, there can be high energy doses at other locations depending on the material and height of the packaged wound dressing, and hence high DUR values overall. The DUR is usually between 1.1 and 10.0. An advantageous DUR range in the context of the invention is at values between 1.0 and 2.0. The less desirable high DUR values occur at low energies in particular. Comparatively high energies, for example 280 keV, are required to obtain an advantageous DUR, especially in the case of products with a higher grammage (island dressings, for example).

This results in the further effect that regions of the product, especially those with a low grammage, are already completely penetrated at these energies. Since the electrons leave the wound dressing again without interacting with the wound dressing, these electrons do not contribute to the sterilization process and reduce the efficiency of the sterilization process.

In experiments, energy values of 250 keV were found to be optimal for a wound dressing with a grammage of 200 g/m².

The sterilization module comprises a shielding device in a preferred embodiment. The shielding device keeps the radiation emitted by the electron beam source away from the surroundings. The shielding device preferably surrounds both the electron beam source and the conveying device. By preference, the sterilization module is self-shielding by virtue of the shielding being brought about by components of the sterilization module. Thus, the shielding is a component of the sterilization module.

According to an advantageous embodiment, the shielding device comprises steel plates. By preference, the steel plates have a height from 5 cm to 32 cm, preferably 10 cm to 27 cm. By preference, the steel plates form an at least partially closed-off hollow body, into the interior of which the electron beam source has been introduced. Alternative materials with a high density or materials containing elements with high atomic numbers also ensure efficient radiation shielding.

Shieldings made of lead, which can be significantly thinner than those made of steel, represent a preferred embodiment. For lead, use is made of a shielding with a thickness of preferably between 5 mm and 80 mm, particularly preferably between 10 mm and 54 mm. The packaged wound dressings are guided through the shielding device to the electron beam source by means of the conveying device. If the packaged wound dressings are transported in the connected pouch chain by means of the conveying device, then the rollers of the conveying device, for example, can be part of the shielding device.

In a preferred embodiment, the connected pouch chain is guided through the sterilization module by means of the conveying device guide. The conveying device guide preferably comprises four rollers in the region of the sterilization module.

The pouch chain is deflected out of the first plane by way of a first roller. The first plane is defined by the pouch chain entering the sterilization module. The pouch chain is steered into the region of the electron beam source in the sterilization module by means of the second and third roller. The packaged wound dressings in the pouch chain are sterilized by means of radiation emanating from the electron beam source. The fourth roller steers the pouch chain out of the second plane after the latter has left the sterilization module. The second plane is a defined by the alignment of the pouch chain between the third and the fourth roller. In this embodiment, the first and the fourth roller are used as part of the shielding device. By way of example, this embodiment is depicted in FIG. 2.

The irradiation of individual products sealed in a primary packaging, a senvisaged by the present invention, was found to be particularly advantageous. As a result of the significantly reduced number of plies in the vertical direction in comparison with the irradiation of a plurality of products in a secondary packaging, it is possible to obtain a good energy dose distribution even at low energies, and this has an advantageous effect on the material compatibility. The acceleration voltage of the sterilization module

$${u}_{S} = \frac{E}{e}$$

with the elementary charge e emerges from the energy of the electrons. The power required for the sterilization can be made available by one or more electron beam sources.

By preference, the current intensity is varied and the acceleration voltage is kept constant in the case of a plurality of electron beam sources. An irradiation of the wound dressing from two or more sides is conceivable if a plurality of electron beam sources are used.

Secondary radiation may occur in the interior of the wound dressing on account of the energies used according to the invention. In the case of thin materials, in particular, secondary radiation may occur on account of back scattering on the conveyor belt of the primary radiation not absorbed by the wound dressing. Thus, a higher energy dose may occur in the interior of the packaged wound dressings in comparison with the surface region facing the radiation source. The backscatter can be used to efficiently irradiate wound dressings with a low grammage (preferably a grammage less than 200 g/m²) using energies greater than 250 keV in particular. Wound dressings with a homogenous structure are particularly suitable. In this embodiment, primary radiation not absorbed by the wound dressing is incident on a metallic reflector and scattered back by the latter.

The conveying device is controlled by a programmable logic controller through open-loop and/or closed-loop mechanisms. For instance, the conveying device can be switched off when a switch-off command is entered into the controller or when the machines are loaded with newsource materials. In a preferred embodiment, the conveying device controller is coupled to the sterilization module controller. For instance, the electron beam source power is matched to the web speed.

The electron beam source can also be stopped quickly in that case, for example if there is a breakdown of the production apparatus or conveying device (for example a material jam). If the packaged wound dressings are transported in a connected pouch chain by means of the conveying device, then the connected packages of the wound dressings are cut to size into individually packaged wound dressings after the sterilization in this embodiment.

### Measuring the Energy Dose During the Irradiation

To determine the actual energy dose applied during the sterilization, a plurality of dosimeters are applied both to the packaging of the wound dressing and in the packaging of the wound dressing. The dosimeters introduced into the packaging of the wound dressing are situated in part in the wound dressing itself and are situated furthest away from the exterior of the packaging of the wound dressing.

The measured values of the dosimeters applied to the exterior of the packaging of the wound dressing serve as comparison values. They are not included in the calculation of the dose uniformity ratio (DUR).

A DUR of 1 represents a homogeneous energy dose over the entire packaged wound dressing. A uniform irradiation over the entire packaged wound dressing is desirable; therefore, a DUR of close to 1 represents a result to be sought after.

### FIGURES

To better understand the present invention and its advantages, reference is now made to the following embodiments in conjunction with the associated figures.

FIG. 1

Island dressings in connected peel pouches.

FIG. 2

Schematic structure of an embodiment of the apparatus for producing a sterilized, packaged wound dressing.

FIGS. 3, 4, 5

Measured values at an acceleration voltage of 200 keV, 250 keV and 280 keV.

FIG. 6

Calculated DUR for the acceleration voltages of 200 keV, 250 keV and 280 keV.

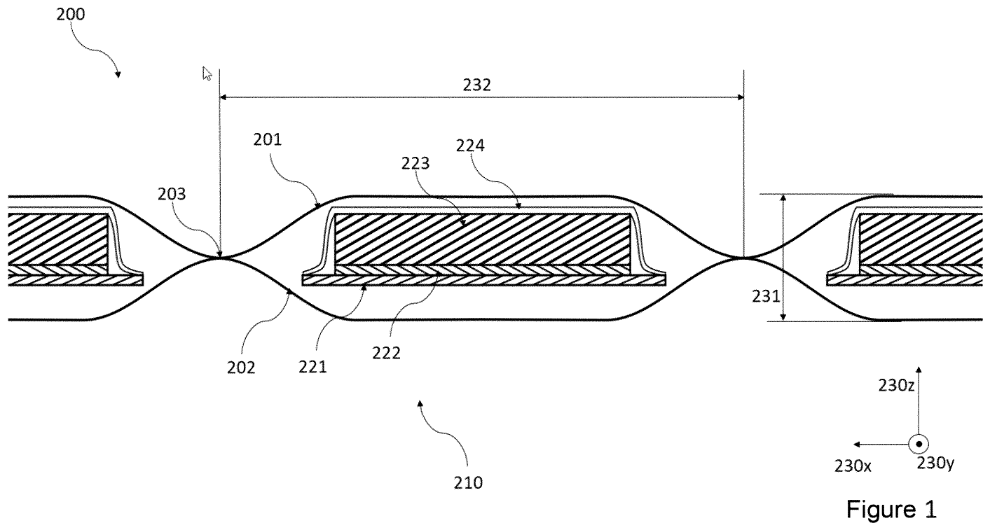

FIG. 1 shows a cross section (not true to scale) through a chain of packaged wound dressings 200. The wound dressing 210 is enclosed by a first material ply 201 and a second material ply 202. The first material ply 201 and the second material ply 202 are connected by means of a seal 203 such that the first material ply 201 and the second material ply 202 form a sealed packaging pouch containing the wound dressing 220. An individual packaged wound dressing 210 is connected to a respective further packaged wound dressing at the seal 203 on respective opposite sides of the packaging pouch. The first material ply 201 and the second material ply 202 extend over the entire length of all packaged wound dressings 200. Together, all packaged wound dressings form a pouch chain 200.

The first material ply 201 consists of a transparent film and the second material ply 202 consists of paper.

The wound dressing 210 comprises an absorbent ply 223. The absorbent ply 223 is situated between the covering layer 224 and the wound contact layer 222. The wound contact layer 222 is completely covered by a backing paper 221. For instance, the covering layer 224 consists of a polyurethane film. The wound contact layer 222, which may have a smaller area than the covering layer 224, may be formed from, for example, a hydrophilic nonwoven material or a perforated polyurethane film coated with an adhesive silicone.

The connected pouch chain is moved in direction 230x by means of a conveying device. The directions 230y and 230z are defined perpendicular to 230x and perpendicular to one another. The direction 230y is aligned along the width of the wound dressing and the direction 230z is aligned along the height of the wound dressing.

In the example shown, the wound dressing with the packaging has a maximum height 231 of 2 mm. The length 232 of the packaged wound dressing is 12 cm in this example. The individual, packaged wound dressing 210 is 8 cm wide. The unpackaged wound dressing has a width of 5 cm and a length of 7 cm. The first material ply 201 and the second material ply 202 are 8 cm wide.

The length of the wound dressing is defined orthogonal to the direction 230x and the width of the wound dressing is defined along 230x.

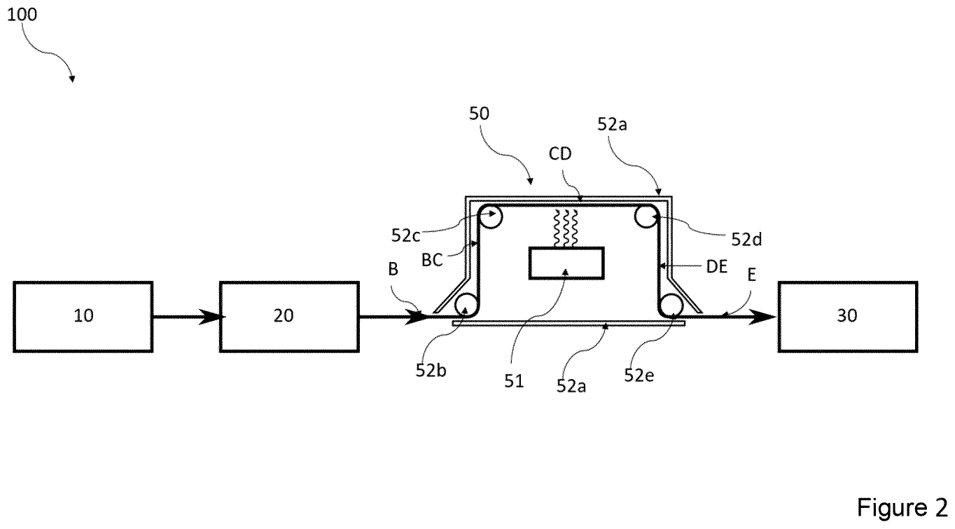

FIG. 2 shows the schematic structure of an apparatus 100 for producing a sterilized, packaged wound dressing. The wound dressing is produced in the production module 10. A conveying device (not shown here) is used to convey the wound dressings to the packaging machine 20, where they are packaged or sealed, with the packaging forming a germ barrier.

The wound dressings are now available in a connected pouch chain as shown in FIG. 1. The connected pouch chain is guided to the sterilization module 50 and, in the latter, guided by the rollers 52b, 52c, 52d, 52e to the electron beam source, which is the electron accelerator 51 in this case. The rollers 52b and 52e are both parts of the shielding device and parts of the conveying device.

The pouch chain is deflected out of the plane B by means of a first roller 52b and then runs in the plane BC. The plane B is defined by the pouch chain entering the sterilization module. The pouch chain runs in the plane BC between the first roller 52b and the second roller 52c. The pouch chain is steered into the region of the electron beam source 51 of the sterilization module 50 by means of the second roller 52c and the third roller 52d and runs in the plane CD. The packaged wound dressings in the pouch chain are sterilized by means of the radiation emanating from the electron beam source 51. After leaving the sterilization module 50, the fourth roller 52e steers the pouch chain into the plane E from the plane DE specified by the alignment of the pouch chain between the third roller 52d and the fourth roller 52e. The first and the fourth roller 52b, 52e are used as component parts of the shielding device.

A further shielding is provided by means of the shielding device 52a. The shielding device 52a consists of lead elements 54 mm thick. After the pouch chain has left the sterilization module 50, it is steered to the cutting-to-size module 30. The cutting-to-size module 30 cuts the 20 connected pouch chain into individual, packaged wound dressings.

In the embodiment of a sterilization module 50 shown in FIG. 2, wound dressings with a maximum height of 2 mm are irradiated. The conveying device speed is 200 m/min. The electron acceleration voltage applied is 250 keV. The power of the electron beam source 51 is 25 6.9 kW, and the current intensity of the electron beam is 27.6 mA. The target value for the energy dose absorbed by the wound dressing is 25 kGy.

The sterilization module 50, depicted only by way of example and for the purpose of illustrating a possible embodiment in FIG. 2, can have a deviating structure within the scope of the 30 present invention. Likewise, the irradiation of the wound dressings to be sterilized can be performed with different parameters within the scope of the ranges proposed according to the invention. In this respect, the embodiment shown in FIG. 2 represents only one variant identified as advantageous by the inventors of the present invention.

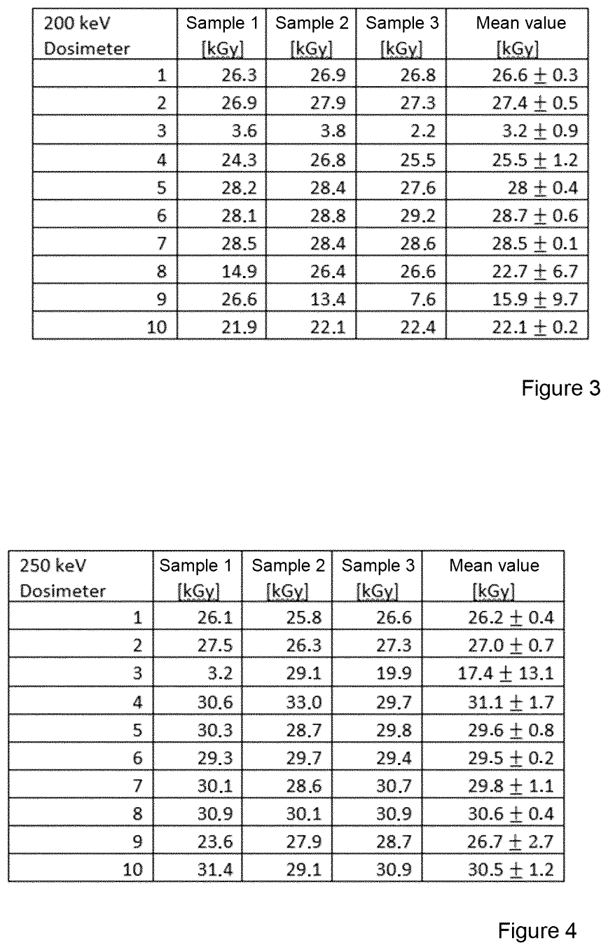

The measured values reproduced in FIGS. 3 to 5 were recorded using the method described below.

Respectively 2 dosimeters are applied to the front and back exterior of the packaging. The measured values of these dosimeters 1, 2, 3, 4 are not included in the DUR calculation. 1 and 2 are fastened to the front exterior, and 3 and 4 are fastened to the back exterior.

### Dosimeters

5,6 and 7 are fastened to the inside of the packaging, between the front exterior of the packaging and the wound dressing. Dosimeters 8 and 9 are introduced into the packaging and into the wound dressing itself. Dosimeter 10 is fastened between the back exterior of the packaging and the wound dressing.

The packaged wound dressing is irradiated from one side. The electron radiation is incident first on the front exterior where dosimeters 1 and 2 have been applied.

The measurements are performed for an acceleration voltage of the sterilization module u₅of 200 keV and a current intensity of 5.6 mA, an acceleration voltage of 250 keV and a current intensity of 6.9 mA, and an acceleration voltage of 280 keV and a current intensity of 7.5 mA.

The target value for the energy dose absorbed by the wound dressing is 25 kGy.

The packaged wound dressing has a height of 2 mm.

The packaged wound dressing is now conveyed through the irradiation region of the sterilization module at a speed of v_{w}=10 m/min by way of the conveying device.

3 measurements are performed for each acceleration voltage.

To avoid backscatter, the packaged wound dressings are placed on a piece of paper 2 mm thick. The paper is situated on the side facing away from the electron beam source.

The measurement results for the acceleration voltage of 200 keV are shown in FIG. 3. The measurement results for the acceleration voltage of 250 keV are depicted in FIG. 4, and the measurement results for the acceleration voltage of 280 keV can be found in FIG. 5.

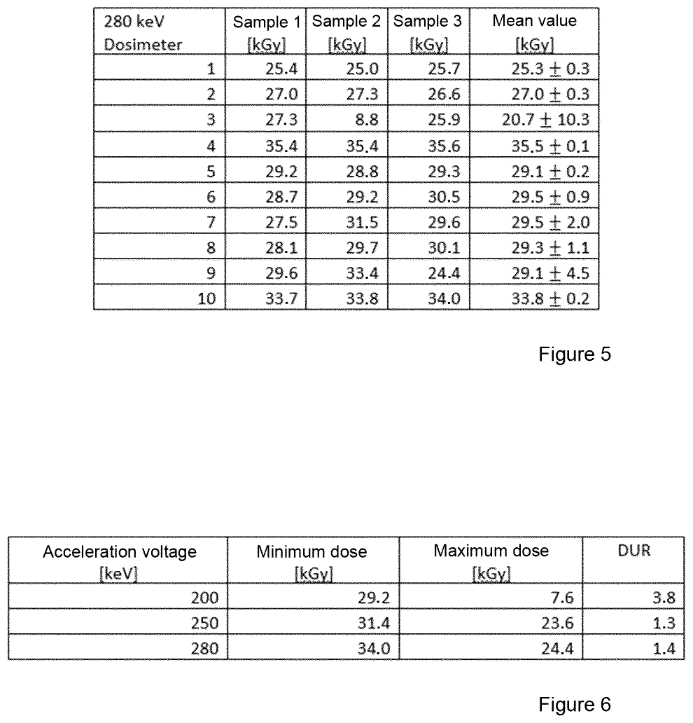

The DUR for all acceleration voltages is calculated in FIG. 6. The highest energy dose and thelowestenergydoseisselectedforeachaccelerationvoltagefromallmeasurementsbelonging to dosimeters 5 to 10. Then, the highest energy dose is divided by the lowest energy dose in order to obtain the DUR value.

A DUR of 3.83 emerges for the acceleration voltage of 200 keV. The irradiation is more uniform for the acceleration voltage of 280 keV. The DUR isat1.39 for the acceleration voltage of 280 keV. The irradiation is most uniform for the acceleration voltage of 250 keV.

The DUR is 1.33 for the acceleration voltage of 250 keV.

It was found that an acceleration voltage of 250 keV is particularly suitable.

1. A method of in-line sterilization of a packaged wound dressing, comprising the following steps:
providing a packaged wound dressing by means of a production apparatus (10, 20),
conveying the packaged wound dressing by means of a conveying device to a sterilization module (50),
sterilizing the packaged wound dressing by means of at least one electron beam source (51) present in the sterilization module (50), wherein the sterilization module (50) is coupled in-line to the production apparatus (10, 20),
a) and wherein the energy dose to which the packaged wound dressings are exposed is at least 15 kGy and no more than 30 kGy,
b) and wherein the energy of the electron radiation is between 150 keV and 350 keV,
c) and wherein the conveying device moves the packaged wound dressings at a speed of at least 10 m/min and no more than 200 min/m.

2. The method of claim 1, wherein there is a plurality of packaged wound dressings which are conveyed in packagings that are connected to one another at their ends to the sterilization module (50) and in that the wound dressing packagings that are connected to one another at their ends are cut to size into individually packaged wound dressings post sterilization.

3. The method of claim 1, wherein there is a plurality of packagings that are connected to one another at their ends and are cut to size into individually packaged wound dressings prior to the sterilization and the individually packaged wound dressings are conveyed to the sterilization module (50).

4. The method of claim 3, wherein the individually packaged wound dressings are held on the conveying device by means of negative pressure.

5. The method of claim 3, wherein the individually packaged wound dressings are held on the conveying device by means of pockets which are sealed or partially opened.

6. The method a of claim 1, wherein the sterilization module (50) has a self-shielding configuration by virtue of the shielding being brought about by components of the sterilization module (50).

7. The method a of claim 1, wherein the conveying device is controlled by a programmable logic controller through open-loop and/or closed-loop mechanisms.

8. The method of claim 1, wherein the wound dressing has a height of from about 0.5 mm to about 10.0 mm.

9. The method of claim 1, wherein the packaged wound dressing has a mean grammage of between from about 0.2
kg/m2 kg/m2
to about 0.5
kg/m2 kg/m2.

10. The method of claim 1, wherein the packaging of the packaged wound dressing is a peel pouch consisting of a first sterile barrier (201) and a second sterile barrier (202), wherein the first sterile barrier (201) is connected to the second sterile barrier (202) by way of an adhering outer region (203) so as to form an internal cavity for accommodating the wound dressing.
